(19) United States
(12) Patent Application Publication
(10) Pub. No.: US 2021/0260096 A1
(43) Pub. Date: Aug. 26, 2021

(54) PHARMACEUTICAL COMPOSITION FOR TREATING WOUND OR ACTIVATING SKIN, CONTAINING BETA-GLUCAN, GLYCITIN, AND 4',6,7-TRIMETHOXYISOFLAVONE

(71) Applicant: Quegen Biotech Co., Ltd., Gyeonggi-do (KR)

(72) Inventors: Eun Ji PARK, Gwangju (KR); Jong Dae LEE, Seoul (KR); Sung Hong KIM, Seoul (KR); Byeong Hee PARK, Gyeonggi-do (KR); Young Kyun KIM, Gyeonggi-do (KR)

(73) Assignee: Quegen Biotech Co., Ltd., Gyeonggi-do (KR)

(21) Appl. No.: 17/254,812

(22) PCT Filed: Jun. 21, 2019
(86) PCT No.: PCT/KR2019/007530

§ 371 Date: Dec. 21, 2020

(30) Foreign Application Priority Data
Jun. 22, 2018 (KR) 10-2018-0072306

(51) Int. Cl. A61K 31/716; A61K 31/7048; A61K 31/352; A61K 8/60; A61K 8/49; A61K 47/38; A61K 8/73; A61K 9/70; A61K 47/32; A61P 17/02; A61Q 19/00; A61K 8/02

(52) U.S. Cl. CPC A61K 31/716; A61K 31/7048; A61K 31/352; A61K 8/60; A61K 8/498; A61K 8/0208; A61K 8/731; A61K 9/7023; A61K 47/32; A61P 17/02; A61Q 19/00; A61K 47/38

(57) ABSTRACT

The present invention relates to a pharmaceutical composition for treating a wound or activating the skin, containing beta-glucan, glycitin (4′-hydroxy-6-methoxyisoflavone-7-D-glucoside), and 4′,6,7-trimethoxyisoflavone.

### TECHNICAL FIELD

This application claims priority to and the benefit of Korean Patent Application No. 10-2018-0072306, filed on Jun. 22, 2018, the disclosure of which is incorporated herein by reference in its entirety.

The present invention relates to a pharmaceutical composition for healing a wound or activating skin, which includes beta (β)-glucan, 4′-hydroxy-6-methoxyisoflavone-7-D-glucoside (glycitin) and 4′,6,7-trimethoxyisoflavone (TMF).

### BACKGROUND ART

The skin is one of the organs that play important life-protecting functions such as protection of the human body from an external stimulus, prevention of water loss, temperature regulation and prevention of bacterial invasion. When the skin is damaged by burns or various injuries, such protective functions are lost, resulting in dysfunction, and it is difficult to treat a damaged region due to various side effects caused by water loss and infection with bacteria invading from the outside, or additional side effects such as secondary dysfunction or damage occurs, and if severe, they also affect life extension. Therefore, in order to speed up the healing of wounds and minimize various secondary side effects, it is essential to treat wounds with an appropriate dressing.

A wound is a condition in which the continuity of tissue is destroyed by external pressure, or a defect occurs in some areas. Mild wounds such as simple trauma can be regenerated by a self-repairing function, but severe burns, or intractable wounds such as complex wounds, bed sores and wounds caused by surgical operations are difficult to completely repair. Since the intractable wounds or extensive wounds may leave defects in performing the original function of the tissue, it is essential to quickly treat wound sites and minimize various secondary side effects. Wound healing is known to be approximately twice as fast when wounds are wet rather than dried. Conventionally, to maintain a wet condition, a method of dressing with gauze soaked with a physiological solution or solution containing a disinfectant has been used. However, due to a frequent replacement cycle of dressing, management is cumbersome, and macerated skin is generated since not only the wound site but also the peripheral area is maintained in a moist condition by a physiological solution or disinfectant. In recent years, as well as protecting wounds using wound dressing products containing hydrogel or hydrocolloid, a method of preventing water leakage from the body and absorbing an exudate to maintain a moist environment has been widely used.

β-glucan is a polymer material consisting of glucose, obtained from various sources, and particularly, yeast-derived β-glucan obtained from the cell wall of yeast is widely known as one of the bioactive materials that act on the skin to not only regulate metabolism and a biorhythm but also have a skin protective function imparting activity to the immune system, and thus is widely used in wound healing (Korean Patent No. 10-1109146). Glycitin increases collagen synthesis in fibroblasts, inhibits elastinase activity and significantly inhibits cell aging induced by hydrogen peroxide. In addition, natural single substances extracted from plants, such as TMF and glycitin, promote the proliferation of the dermal layer and collagen synthesis, exhibiting an excellent effect in wrinkle care. However, there has been no research on whether mixing of these three materials in a specific ratio has a synergistic effect in wound healing.

Therefore, the inventors confirmed that when β-glucan, glycitin and TMF are mixed in a specific ratio and then treated, it has a higher wound healing effect than when the three materials are treated alone, and thus the present invention was completed.

### DISCLOSURE

### Technical Problem

The present invention is directed to providing a pharmaceutical composition for healing a wound or activating skin, which includes beta (β)-glucan, 4′-hydroxy-6-methoxyisoflavone-7-D-glucoside (glycitin) and 4′,6,7-trimethoxyisoflavone (TMF).

### Technical Solution

The present invention provides a pharmaceutical composition for healing a wound, which includes β-glucan, glycitin; and TMF.

The present invention also provides a wound healing composition for an external preparation for skin, which includes β-glucan, glycitin and TMF.

The present invention also provides a pharmaceutical composition for healing a wound, which further includes carboxymethyl cellulose (CMC) in the above-described pharmaceutical composition.

The present invention also provides a pharmaceutical composition for healing a wound, which further includes polyvinyl alcohol in the above-described pharmaceutical composition.

The present invention also provides a cosmetic composition for alleviating a wound, which includes β-glucan, glycitin and TMF.

The present invention also provides a wound healing method, which includes administering β-glucan, glycitin and TMF to a subject in need of treatment at a pharmaceutically effective amount.

The present invention also provides a use of β-glucan, glycitin and TMF in the composition for healing a wound’.

### Advantageous Effects

An external preparation for skin of the present invention is prepared by mixing beta (β)-glucan, 4′-hydroxy-6-methoxyisoflavone-7-D-glucoside (glycitin) and 4′,6,7-trimethoxyisoflavone (TMF) at specific concentrations, and as a result of treating a wound site with the external preparation for skin, a superior wound healing effect than when each component is treated individually can be exhibited. Accordingly, it is possible to accelerate the speed of wound healing in patients in recovery after surgery or with a wound, protect an injury and provide a moist environment. Further, by developing an external preparation for skin further including carboxymethyl cellulose (CMC) or polyvinyl alcohol, a hydrogel tube-type external preparation for skin and a hydrocolloid patch-type external preparation for skin, which perform a function of absorbing a wound-derived exudate, is provided.

### DESCRIPTION OF DRAWINGS

FIG. 1 is the results showing the differentiation and migration of human keratinocytes and fibroblasts according to the concentration of beta (β)-glucan.

FIG. 2 is the results showing factor expression effects of epithelial cells and fibroblasts according to 0.5% β-glucan treatment, confirmed by western blotting.

FIG. 3 is the results showing the differentiation, migration and growth factor expression of epithelial cells and fibroblasts according to concentrations and mixtures of 4′-hydroxy-6-methoxyisoflavone-7-D-glucoside (glycitin) and 4′,6,7-trimethoxyisoflavone (TMF), confirmed by western blotting.

FIG. 4 is the results of the differentiation and migration of keratinocytes according to a mixing ratio of glycitin and TMF.

FIG. 5 is the results of growth factor expression in a mixed solution of glycitin and TMF in the single culture and the co-culture of epithelial cells and fibroblasts, confirmed by western blotting.

FIG. 6 is the results showing the wound healing effect of a hydrogel for wound healing, which includes β-glucan, carboxymethyl cellulose (CMC), TMF and glycitin.

FIG. 7 is the result showing the swelling ratios of hydrocolloid patches, which include β-glucan, polyvinyl alcohol, TMF and glycitin, according to a polyvinyl alcohol weight.

FIG. 8 is the results showing the wound healing effects of hydrocolloid patches, which include β-glucan, polyvinyl alcohol, glycitin and TMF.

### MODES OF THE INVENTION

When beta (β)-glucan, 4′-hydroxy-6-methoxyisoflavone-7-D-glucoside (glycitin) and 4′,6,7-trimethoxyisoflavone (TMF) are mixed in a specific ratio and then treated, it was confirmed that a higher wound healing effect than when these three materials are treated alone is exhibited, and thus the present invention was completed.

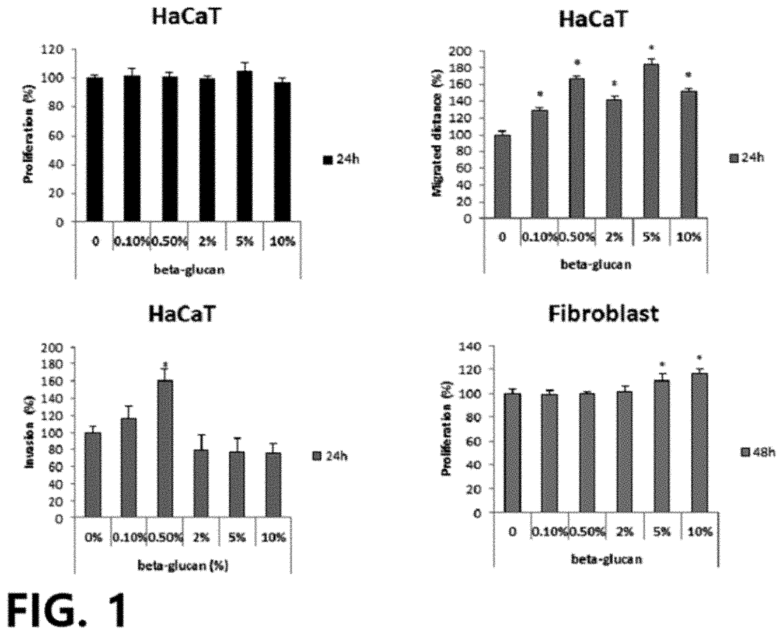

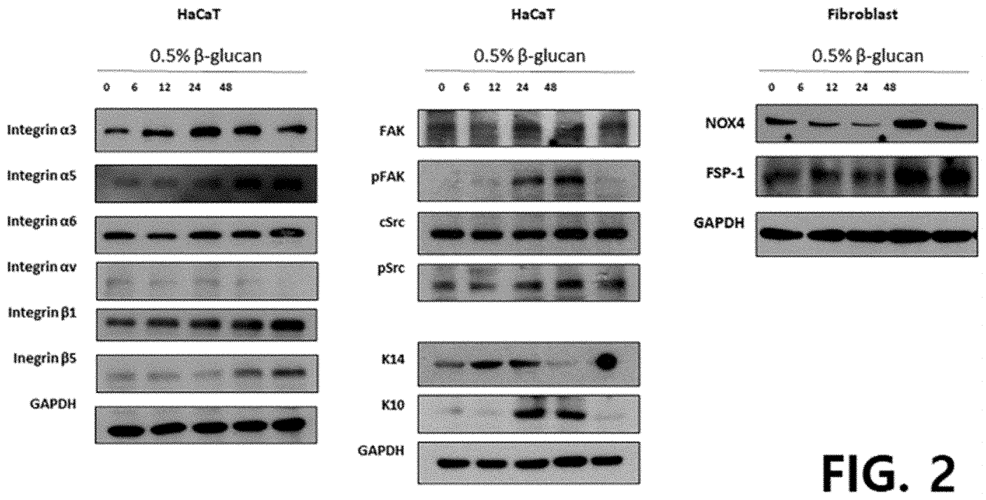

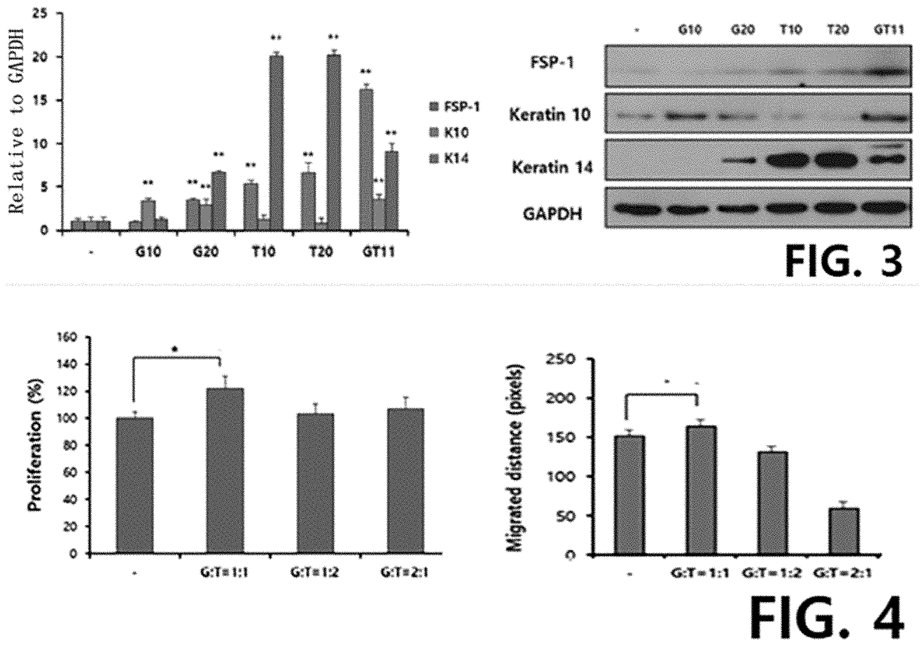

The present invention showed that, as a result of testing a concentration which exhibits a β-glucan effect, 0.5% β-glucan has an effect in differentiation, migration and invasion of human keratinocytes, and also has an excellent effect in differentiation of fibroblasts (FIG. 1). When 0.5% β-glucan is treated, it was confirmed that the expression of growth-inducing factors in the epithelial cells and fibroblasts also effectively increases (FIG. 2). The inventors treated glycitin and TMF individually at different concentrations and a 1.43:1 mixture of glycitin and TMF to confirm a synergistic effect of the glycitin and the TMF. As a result, they confirmed that the expression of growth factors in the epithelial cells and the fibroblasts increases (FIG. 3). In addition, as a result of treating epithelial cells with a mixture of glycitin and TMF at different ratios such as 1.43:1, 2.86:1 or 1.43:2, in the case of the 1.43:1 mixture of the glycitin and the TMF, it can be confirmed that the differentiation and migration of the epithelial cells increased (FIG. 4). As a result of co-culturing epithelial cells and fibroblasts and treating them with a mixed solution of glycitin and TMF in a ratio of 1.43:1, it was confirmed that the expression of growth factors in the epithelial cells and the fibroblasts increase.

The present invention provides a pharmaceutical composition for healing a wound, which includes β-glucan, glycitin and TMF.

The β-glucan may be represented by Formula 1 below:

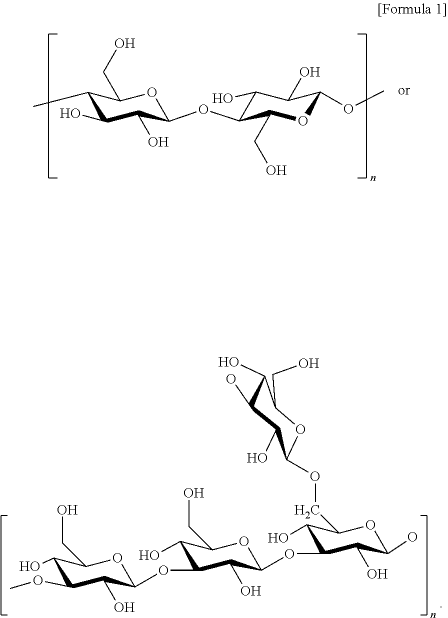

The glycitin may be represented by Formula 2 below.

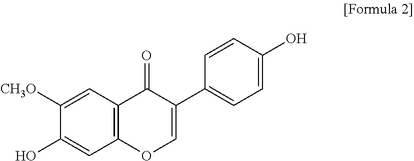

The TMF may be represented by Formula 3 below:

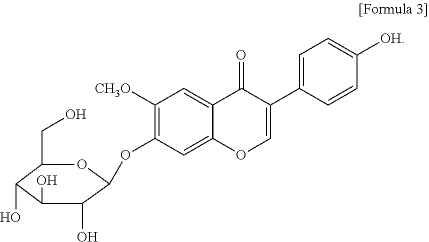

The inventors attempted to confirm the effects of ratios of β-glucan, glycitin and TMF on the differentiation and migration of epithelial cells and fibroblasts according to a mixing ratio, and determined that the mixed solution of 0.25 w/v % of β-glucan, 0.00446 w/v % of glycitin and 0.00312 w/v % of TMF showed the highest effect (Table 1).

The weight ratio of the β-glucan, glycitin and TMF may be 320.5:1.43:1 to 1602:1.43:1.

The pharmaceutical composition may include β-glucan at 0.1 to 5.0 w/v %, preferably, 0.1 to 0.5 w/v %, and most preferably 0.25 w/v %.

The pharmaceutical composition may include glycitin at 0.00223 to 0.0223 w/v %, preferably, 0.00223 to 0.00446 w/v %, and most preferably 0.00446 w/v %.

The pharmaceutical composition may include TMF at 0.00156 to 0.0156 w/v %, preferably, 0.00156 to 0.00312 w/v %, and most preferably, 0.00312 w/v %.

The glycitin and the TMF may be mixed in a ratio of 1.43:2 to 2.86:1, and preferably, 1.43:1.

The present invention provides a wound healing composition for an external preparation for skin, which includes β-glucan, glycitin and TMF.

The composition for an external preparation for skin according to the present invention may be formulated to contain a cosmetically or dermatologically acceptable medium or base. The composition may be prepared in any one of all formulations suitable for topical application, which is selected from the group consisting of a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, a body essence, a makeup base, a foundation, a hair dye, a shampoo, a rinse, a body cleanser, a toothpaste, a mouthwash, an ointment, a tube, a patch and a spray. This composition may be prepared by a conventional method known in the art.

In addition, the composition of the present invention may contain an additive conventionally used in the cosmetic or dermatological field, such as a lipid, an organic solvent, a solubilizer, a concentrate, a gelling agent, a softening agent, an antioxidant, an emulsifier, a stabilizer, a foaming agent, a fragrance, a surfactant, water, an ionic or non-ionic emulsifier, a filler, a sequestering agent, a chelating agent, a preservative, a vitamin, a blocking agent, a wetting agent, an essential oil, a dye, a pigment, a hydrophilic or lipophilic active agent, a lipid vesicle or any other ingredient conventionally used in cosmetics. The additive is introduced at an amount generally used in the cosmetic or dermatological field.

The present invention provides a composition for an external preparation for skin, which further includes carboxymethyl cellulose (CMC) in the above-described composition for an external preparation for skin.

The inventors studied a hydrogel tube-type external preparation for skin which performs a function of absorbing a wound-derived exudate by protecting an injury and providing a moist environment. The present invention provides a composition for an external preparation for skin, which further includes polyvinyl alcohol in the above-described composition for an external preparation for skin. A wound healing hydrogel was prepared by adding 4 wt % of CMC (Mw: ˜250,000) to a 0.25% β-glucan solution, mixing the resulting solution by stirring at 60° C., adding TMF and glycitin at 0.00312 w/v % and 0.00446 w/v %, respectively, and putting the mixed aqueous solution into a mold. As a result of measuring viscosity according to wt % of CMC, it was determined that the viscosity of a hydrogel containing 4 wt % CMC is suitable for preparing the hydrogel. The wound healing effect of the prepared hydrogel tube type was confirmed. Five ICR mice were assigned to each experimental group, and the back of each mouse was injured with a 5-mm biopsy punch, and then treated with two different wound covering materials, a non-treated group (control) or a physiological saline-treated group, followed by monitoring the change in wounds for 14 days and evaluating the efficiency of the composition from a wound recovery rate and a tissue test. As a result, it was demonstrated that the CMC composition containing β-glucan, glycitin and TMF as active ingredients has the highest effect.

The CMC may be contained at 0 to 8 w/v %, preferably, 2 to 6 w/v %, and more preferably, 4 w/v % with respect to the total parts by weight of the composition.

The composition may be prepared in any one formulation selected from the group consisting of a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, a body essence, a makeup base, a foundation, a hair dye, a shampoo, a rinse, a body cleanser, a toothpaste, a mouthwash, an ointment, a tube, a patch, a gel and a spray, and most preferably, a gel.

The inventors of the present invention developed a hydrogel tube-type external preparation for skin which performs a function of absorbing a wound-derived exudate by protecting a wound and providing a moist environment. Further, a composition for a patch-type external preparation for skin, which includes polyvinyl alcohol in the composition for an external preparation for skin, was studied. 5, 7 or 10 wt % of polyvinyl alcohol (PVA, Mw: 85,000 to 124,000) was added to distilled water and then mixed by stirring at 80° C. The mixed solution was molded in a polystyrene dish with a diameter of 120 mm, frozen at −80° C., and thawed at room temperature. This process was repeated twice to perform physical crosslinking of PVA and thus evaluate physical properties and prepare a hydrocolloid-type patch. Subsequently, a wound healing hydrocolloid was prepared by coating the hydrocolloid surface with a solution prepared by adding TMF and glycitin to a 0.25% β-glucan solution at 0.00312 w/v % and 0.00446 w/v %, respectively. As a result of measuring the degree of swelling according to wt % of PVA, it was determined that 7 wt % of the hydrocolloid is more likely to absorb an exudate from wounded skin. A comparative experiment was performed to determine wound healing efficiency of the prepared hydrocolloid patch-type wound covering materials. Five ICR mice were assigned to each experimental group, and the back of each mouse was injured with a 5-mm biopsy punch, and then treated with the prepared wound covering patch, a non-treated group (control), a physiological saline gauze-treated group or a common bandage-treated group, followed by monitoring the change in wounds for 14 days. Efficiency was evaluated from a wound recovery rate, and it was confirmed that a PVA patch composition containing β-glucan, glycitin and TMF as active ingredients exhibited the highest effect.

The PVA was contained at 5 to 10 w/v %, preferably, 6 to 8 w/v %, and more preferably, 7 w/v % with respect to the total parts by weight of the composition.

The composition may be prepared in any one formulation selected from the group consisting of a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, a body essence, a makeup base, a foundation, a hair dye, a shampoo, a rinse, a body cleanser, a toothpaste, a mouthwash, an ointment, a tube, a patch, a gel and a spray, and most preferably, a patch.

The wound may be a burn, an ulcer, a trauma, a post-surgical wound, a post-delivery wound, a chronic wound or a wound caused by dermatitis.

A cosmetic composition for alleviating a wound, which includes β-glucan, glycitin and TMF, is provided.

The present invention provides a method of treating a wound, which includes administering β-glucan, glycitin and TMF to a subject in need of treatment at a pharmaceutically effective amount.

The “subject” used herein refers to refers to a target in need of treatment of a disease, and more specifically, all animals including a primate including a human, a cow, a horse, sheep, a pig, a chicken, a turkey, a quail, a cat, a dog, a mouse, a rat, a rabbit and a guinea pig. The disease may be effectively prevented or treated by administering the pharmaceutical composition of the present invention to the subject. The pharmaceutical composition of the present invention may be administered in combination with a conventional therapeutic agent.

The present invention provides a use of β-glucan, glycitin and TMF for use in a wound healing composition.

The cosmetic composition may be prepared in any one formulation selected from the group consisting of a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, a body essence, a makeup base, a foundation, a hair dye, a shampoo, a rinse, a body cleanser, a toothpaste, and a mouthwash.

Hereinafter, to help in understanding the present invention, exemplary examples will be suggested. However, the following examples are merely provided to more easily understand the present invention, and not to limit the present invention.

### Experimental Example 1. Effect of β-Glucan in Epithelial Cells and Fibroblasts

The differentiation, migration and invasion of epithelial cells, such as human keratinocytes, and the differentiation of fibroblasts according to the single treatment of β-glucan were examined. β-glucan was produced by QUEGEN BIOTECH Co., LTD and used. Human dermal fibroblasts and human keratinocytes were purchased from Korean Cell Line Bank and used. The human dermal fibroblasts (HDFs) or the human keratinocytes (HaCaTs) were seeded in a 6-well plate containing 12 mL of 10% FBS-containing DMEM at 1×10⁴ cells/well (HDF) and 5×10⁴ cells/well (HaCaT), and cultured under conditions of 37° C. and 5% CO₂. During culture, the concentration of the epithelial cells (HaCaTs) and the fibroblasts were treated with 200 μL of β-glucan to have a concentration of 0 to 10% in a concentration dependent manner. By confirming the differentiation, migration and invasion of epithelial cells by concentration, the optimal results were confirmed from 0.5% β-glucan, and no significant change in differentiation of the fibroblasts was observed (FIG. 1). In addition, in the epithelial cells and the fibroblasts treated with the 0.5% β-glucan solution, the expression of various growth factors and proteins was examined by western blotting, confirming a significant change in the epithelial cells (FIG. 2).

### Experimental Example 2. Effects of Glycitin and TMF in Fibroblasts

Effects of glycitin and TMF in inducing the differentiation of epithelial cells and fibroblasts were examined. Glycitin (10 μM (0.00446 w/v %) and 20 μM (0.00892 w/v %)), TMF (10 μM (0.00312 w/v %) and 20 μM (0.006242 w/v %)) or a 1:1 mixture of glycitin (10 μM (0.00446 w/v %)) and TMF (10 μM (0.00312 w/v %)) was prepared. Human dermal fibroblasts (HDFs) or human keratinocytes (HaCaTs) were seeded in a 6-well plate containing 12 mL of 10% FBS-containing DMEM at 1×10⁴ cells/well (HDF) and 5×10⁴ cells/well (HaCaT), and cultured under conditions of 37° C. and 5% CO₂. The fibroblasts or the epithelial cells were treated with each of the glycitin, TMF, and the mixed solution of glycitin and TMF, and the change in expressed protein of each type of cell in the cell culture was observed by western blotting. Compared with when glycitin or TMF was used alone, when these two materials were mixed at an equal ratio (1:1), it was confirmed that the differentiation of the epithelial cells and the fibroblasts significantly increased, and factors for inducing the differentiation of each type of cell also increased. It was confirmed that, to increase a wound healing effect, the use of the mixture of these two materials is effective (FIG. 3).

### Experimental Example 3. Effect of Mixing Ratio of Glycitin and TMF in Epithelial Cells

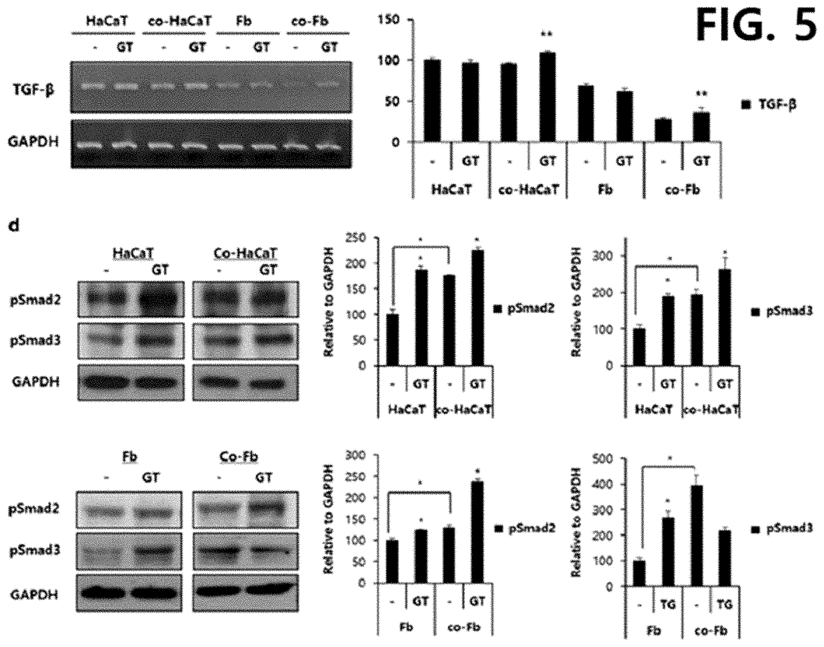

In addition, samples were prepared by changing the mixing ratios of glycitin and TMF to 1.43:1, 2.86:1 and 1.43:2, and human keratinocytes (HaCaTs) were seeded in a 6-well plate containing 12 mL of 10% FBS-containing DMEM at 5×10⁴ cells/well (HaCaT), and then the samples were treated under conditions of 37° C. and 5% CO₂, and then incubated for 24 hours, followed by examining cell differentiation and migration. When glycitin and TMF were mixed at 1.43:1, it was confirmed that the mixture exhibits an excellent effect on the differentiation and migration of epithelial cells (FIG. 4). Samples were prepared by changing the mixing ratio of glycitin and TMF, and using an experimental group in which 5×10⁴ cells/well of HaCaTs were incubated in an upper portion of a Transwell invasion kit containing 12 mL of 10% FBS-containing DMEM under conditions of 37° C. and 5% CO₂ for 24 hours, 5×10⁴ cells/well of HaCaTs were seeded in the upper portion and 1×10⁴ cells/well of HDFs were seeded in the lower portion of a Transwell invasion kit, 24 mL of 10% FBS-containing DMEM and the samples were treated and then incubated under conditions of 37° C. and 5% CO² for 24 hours, followed by confirming the induction of growth factors. It was confirmed that the mixture of glycitin and TMF promotes the proliferation of the epithelial cells and the fibroblasts (FIG. 5).

### Experimental Example 4. Composite Formulation of β-Glucan, Glycitin and TMF

β-glucan was used as solution A, and a 1.43:1 mixture of glycitin/TMF was used as solution B, and these solutions were used alone or in combination at concentrations as shown in Table 1 below, thereby preparing test solutions. 5×10⁴ cells/well of HaCaTs were seeded in the upper part and 1×10⁴ cells/well of HDFs were seeded in the lower part of a Transwell invasion kit, 24 mL of 10% FBS-containing DMEM and the three kinds of test solutions were treated and incubated under conditions of 37° C. and 5% CO₂ for 24 hours, followed by determining an optimal concentration condition.

TABLE 1
Wound healing effect according to composition of mixed solution of
β-glucan/glycitin/TMF (Alleviation rate (%) with respect to control PBS)

| | | Proliferation (%) | Migration (%) |
|---|---|---|---|
| β-glucan | 0.1% | 105 | 110 |
| (Solution A) | 0.25% | 111 | 130 |
| | 0.5% | 120 | 151 |
| | 0.7% | 125 | 153 |
| | 1.0% | 122 | 161 |
| | 1.5% | 118 | 110 |
| TMF + | 0.000446% + | 115 | 101 |
| Glycitin | 0.000312% | | |
| (Solution B) | 0.00223% + 0.00156% | 130 | 106 |
| | 0.00446% + 0.00312% | 145 | 105 |
| | 0.00892% + 0.00624% | 147 | 107 |
| | 0.0223% + 0.0156% | 160 | 120 |
| | 0.0446% + 0.0312% | 152 | 109 |
| Mixed | 0.25% + (0.00223% + | 175 | 163 |
| composition | 0.00156%) | | |
| (Solution A + | 0.25% + (0.00446% + | 220 | 185 |
| Solution B) | 0.00312%) | | |
| | 0.25% + (0.0223% + | 186 | 156 |
| | 0.0156%) | | |
| | 0.25% + (0.0446% + | 147 | 140 |
| | 0.0312%) | | |
| | 0.5% + (0.00446% + | 115 | 133 |
| | 0.00312%) | | |
| | 0.5% + (0.0446% + | 110 | 130 |
| | 0.0312%) | | |
| | 1.0% + (0.0446% + | 101 | 103 |
| | 0.0312%) | | |

When a pharmaceutical compound is prepared by mixing single materials under an optimal composition condition, a mechanism in tissue to prevent excessive proliferation of wound tissue and maintain homeostasis is operated. Accordingly, the optimal composition condition was established under a sub-optimal condition, and thus the optimal composition was confirmed. Particularly, a composition prepared by mixing 0.25% β-glucan, 0.00312% TMF (10 μM) and 0.00446% glycitin (10 μM) showed the synergistic effect of higher proliferation and migration than when these three materials were used individually.

### Experimental Example 5. Measurement of Viscosity of Wound Healing Hydrogel Containing β-Glucan, TMF and Glycitin

A 100 mL beaker was filled with the hydrogel prepared in Experimental Example 4, and then viscosity was measured using a viscometer. The measurement was performed using a #64 spindle at 12 rpm, and a measurement value was confirmed at 53.5% torque. When a CMC content was 4 wt %, compared with 0 wt %, the cP value was higher, and it was determined that the cP value of 4 wt % is suitable (Table 2).

TABLE 2

| | Concentration (%) | |
|---|---|---|
| β-glucan | 0.25% | |
| CMC | 0% | 4% |
| Viscosity (cP) | 830 ± 50 | 26990 ± 100 |

### Experimental Example 6. Wound Healing Effect of Wound Healing Hydrogel Containing β-Glucan, TMF and Glycitin

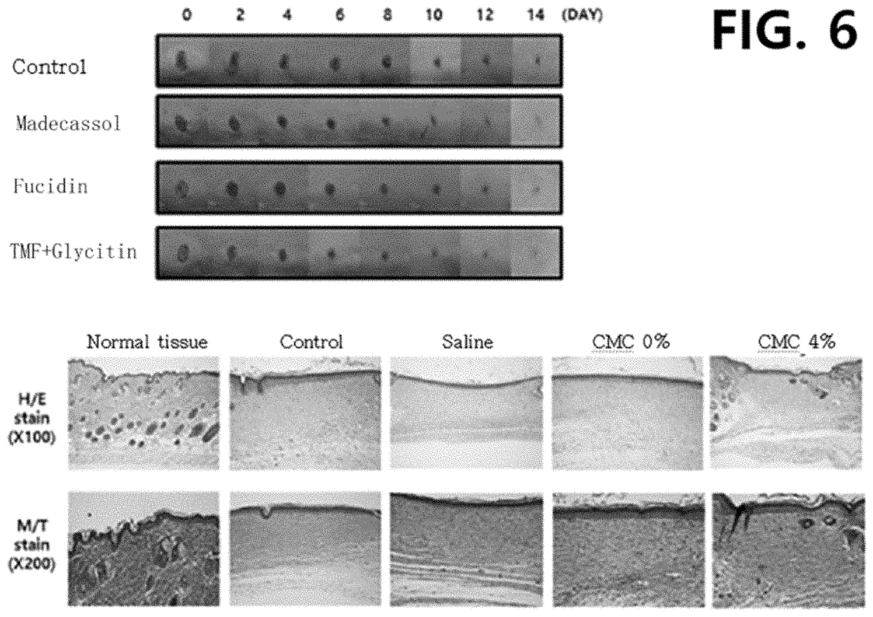

Using each of the hydrogel wound covering materials prepared in Experimental Example 4, wound healing efficiencies were compared. Five ICR mice were assigned to each experimental group, and the back of each mouse was injured with a 5-mm biopsy punch and then treated with each of two types of wound covering materials, a non-treated group (control) and a physiological saline-treated group, followed by monitoring the change in wounds for 14 days. Efficiency was evaluated from the recovery rate of a wound and a biopsy, and it was confirmed that a CMC composition containing β-glucan, glycitin and TMF as active ingredients exhibits the highest effect (FIG. 6).

### Experimental Example 7. Preparation of Wound Healing Hydrocolloid Patch Containing β-Glucan, TMF and Glycitin

5, 7 or 10 wt % of PVA (Mw. 85,000˜124,000) was added to distilled water, and mixed by stirring at 80° C. The mixed solution was molded in a polystyrene dish with a diameter of 120 mm, frozen at −80° C., and thawed at room temperature. This process was repeated twice to perform physical crosslinking of PVA and thus evaluate physical properties and prepare a hydrocolloid-type patch. Subsequently, a wound healing hydrocolloid was prepared by coating the hydrocolloid surface with a solution prepared by adding TMF and glycitin to a 0.25% β-glucan solution at 0.00312 w/v % (10 μM) and 0.00446 w/v % (10 μM), respectively.

### Experimental Example 8. Measurement of Degree of Swelling of Wound Healing Hydrocolloid Patch Containing β-Glucan, TMF and Glycitin

The hydrocolloid prepared in Example 5 was cut to a size of 5×5 cm, and put on a weighing dish to measure a weight (W1). 37° C. PBS was added to the hydrocolloid whose initial weight had been measured in consideration of an absorption capability. After standing for 30 minutes, a later weight (W2) of the sample was measured. The degree of swelling of the hydrocolloid was calculated by the following equation.

$$Degree of swelling ( \% ) = \frac{( w 2 - w 1 )}{w 1} \times 100$$

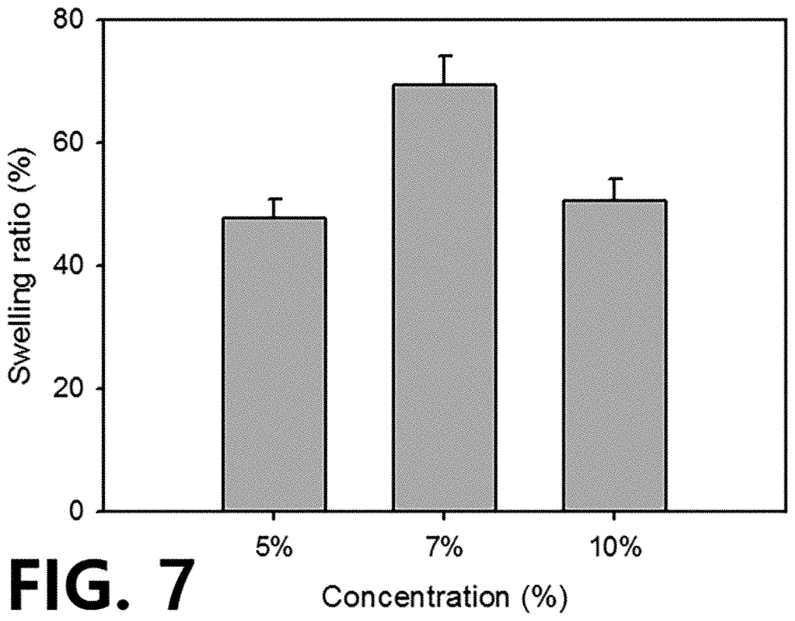

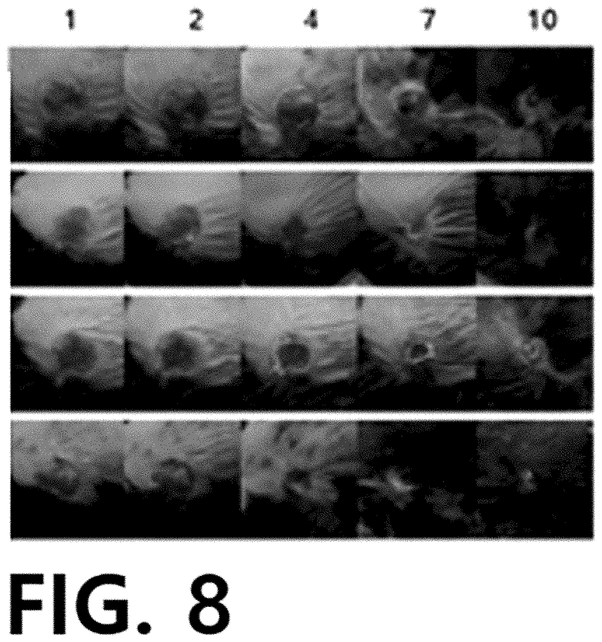

As shown in FIG. 8, the degree of swelling of the PVA hydrocolloid was approximately 47% when the hydrocolloid content was 5 wt %, showed a higher value of approximately 80% when the hydrocolloid content was 7 wt %, demonstrating that 7 wt % of the hydrocolloid more easily absorbs an exudate from wounded skin (FIG. 7).

### Experimental Example 9. Wound Healing Effect of Wound Healing Hydrocolloid Patch Containing β-Glucan, TMF and Glycitin

Using the prepared hydrocolloid patch-type wound covering material, wound healing efficiencies were compared. Five ICR mice were assigned to each experimental group, and the back of each mouse was injured with a 5-mm biopsy punch, and then treated with the prepared wound covering material patch, a non-treated group (control), a physiological saline gauze-treated group, and a common bandage-treated group, followed by monitoring the change in wounds for 10 days. Efficiency was evaluated from a wound recovery rate, and it was confirmed that a PVA patch composition containing β-glucan, glycitin and TMF as active ingredients exhibits the highest effect (FIG. 8).

1. A pharmaceutical composition for healing a wound, comprising:
beta (β)-glucan, 4′-hydroxy-6-methoxyisoflavone-7-D-glucoside (glycitin) and 4′,6,7-trimethoxyisoflavone (TMF).

2. The pharmaceutical composition of claim 1, wherein a weight ratio of the β-glucan, glycitin and TMF is 320.5:1.43:1 to 1602:1.43:1.

3. The pharmaceutical composition of claim 1, wherein the β-glucan is contained at 0.1 to 5.0 wt %.

4. The pharmaceutical composition of claim 1, wherein the glycitin is contained at 0.00223 to 0.0223 wt %.

5. The pharmaceutical composition of claim 1, wherein the TMF is contained at 0.00153 to 0.0156 wt %.

6. A wound healing composition for an external preparation for skin, comprising: beta (β)-glucan, 4′-hydroxy-6-methoxyisoflavone-7-D-glucoside (glycitin) and 4′,6,7-trimethoxyisoflavone (TMF).

7. The wound healing composition of claim 6, which is formulated in any one selected from the group consisting of an ointment, a tube, a patch, a gel and a spray.

8. A pharmaceutical composition for healing a wound, further comprising carboxymethyl cellulose (CMC), in addition to the pharmaceutical composition of claim 1.

9. A pharmaceutical composition for healing a wound, further comprising polyvinyl alcohol (PVA), in addition to the pharmaceutical composition of claim 1.

10. The pharmaceutical composition of claim 9, wherein the wound is a burn, an ulcer, a trauma, a post-surgical wound, a post-delivery wound, a chronic wound or a wound caused by dermatitis.

11. A cosmetic composition for alleviating a wound, comprising:
beta (β)-glucan, 4′-hydroxy-6-methoxyisoflavone-7-D-glucoside (glycitin) and 4′,6,7-trimethoxyisoflavone (TMF).

12. The cosmetic composition of claim 11, which is formulated in any one selected from the group consisting of a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, a body essence, a makeup base, a foundation, a hair dye, a shampoo, a rinse, a body cleanser, a toothpaste, and a mouthwash.

13. A method of healing a wound, comprising:
administering beta (β)-glucan, 4′-hydroxy-6-methoxyisoflavone-7-D-glucoside (glycitin) and 4′,6,7-trimethoxyisoflavone (TMF) to a subject in need of treatment at a pharmaceutically effective amount.

14. A use of beta (β)-glucan, 4′-hydroxy-6-methoxyisoflavone-7-D-glucoside (glycitin) and 4′,6,7-trimethoxyisoflavone (TMF) for use in a composition for healing a wound.
